(19) United States
(12) Patent Application Publication
(10) Pub. No.: US 2008/0051684 A1
(43) Pub. Date: Feb. 28, 2008

(54) Non-Surgically Correcting Abnormal Knee Loading: Treatment and Training Equipment

(76) Inventor: Kazuyoshi Gamada, Hiroshima (JP)

Correspondence Address: GREENLEE WINNER AND SULLIVAN P C, 4875 PEARL EAST CIRCLE, SUITE 200, BOULDER, CO, 80301, US

(21) Appl. No.: 10/597,873

(22) PCT Filed: Feb. 10, 2005
(86) PCT No.: PCT/US05/04127

§ 371 Date: Jul. 16, 2007

Related U.S. Application Data
(60) Provisional application No. 60/543,515, filed on Feb. 10, 2004.

(51) Int. Cl. A61F 5/00

(52) U.S. Cl. 602/16; 602/26

(57) ABSTRACT

The invention provides a device and a method for exercising a user's leg whose lower leg tibial portion can be rotatably controlled during exercise. The device includes user-controlled rotatable drums connected to a sheet that surrounds a lower leg cuff for rotating and providing corrective forces on a user's knee joint during leg exercise. The device can be used with weight training equipment commonly found in commercial gyms, as well as in a user's home, or in clinics, to assist in rehabilitating malaligned knee joints.

### FIELD OF INVENTION

This invention is in the field of training and treatment equipment, and methods to reduce and correct abnormal knee loading using this equipment.

### BACKGROUND OF THE INVENTION

The knee is composed of three bones, the patella (knee cap), the femur and the tibia. The meniscus (cartilage), composed of the lateral and medial menisci, cushion and distribute the weight of the femur uniformly across the joint. The gaps between the tibia and femur on the inside and outside of the knee are called the “medial” and “lateral” compartments, respectively. The condyle is the smooth, rounded end of the femur that allows the femur to move easily over the surface of the menisci on the tibial plateau. The ligaments and tendons control the motion of the knee joint. The tendons connect the patella; the ACL (anterior cruciate ligament) and PCL (posterior cruciate ligament) prevent the tibia from sliding forward or backward and limit the tibia's rotation; the collateral ligaments minimize side-to-side motion and stabilize the knee.

Because the knee joint sustains the mechanical forces repeatedly throughout one's standing activities, and is one of the most mobile and flexible joints in the body, it is vulnerable to injury and degeneration. The knee is kept in alignment by the tendons and ligaments. Malalignment of the knee joint can occur when any of the tendons or ligaments is damaged. In particular, malalignment occurs when the tibia is translated and rotated relative to the femur. Malalignment of the tibia causes abnormal loading of forces across the knee and osteoarthritis (OA) of the knee. Knee OA is one of the most common orthopedic problems with about six percent of US adults over 30 years of age suffering from this disease. The total cost of knee OA was estimated as $15.5 billion in 1994. Advanced OA often requires surgery to restore leg alignment, physical function, and reduce knee joint pain.

Though the malalignment of the knee is typically seen in valgus (bow-legged) or varus (knocked kneed) deformity, it also involves the rotational malalignment and translation of the tibia. In particular, the rotational and translational malalignment are often observed in the initial stage of knee OA when the valgus or varus deformity is not yet developed, and there is no treatment theory or methodology for correcting these components. Therefore, there is a need for developing a methodology for correcting the rotational malalignment to prevent further deformity.

Treatment for knee injury often involves therapeutic exercise programs with bracing, medication, and/or other physical therapy modalities. However, current treatment regimens for knee exercise and therapy focus on gaining muscle strength and restoring the range of motion. The treatment and training equipment associated with these treatment regimens do not correct the underlying knee malalignment. Thus, there is need in the art for exercise and treatment equipment that can correct knee malalignment while simultaneously strengthening and restoring the knee's range of motion.

### SUMMARY OF THE INVENTION

The present invention helps patients restore the rotational alignment of the malaligned knee to that of a healthy knee, and restore the knee function by regaining the range of motion and strengthening of the knee muscles. Therefore, it can ultimately correct the deformity to the extent allowed by the underlying articular cartilage structural damage. Accordingly, the present invention provides a method of leg exercise that can help treat knee osteoarthritis by simultaneously correcting the knee rotational malalignment and restoring the knee function, which allows for an optimal loading distribution across the knee joint and ultimately minimizes cartilage degeneration.

The present invention is training and treatment equipment comprising a rotation corrector. The rotation corrector is a novel assembly of leg holding device, which can rotate the knee passively without any leg muscle activity or require leg muscle activity to rotate the leg into a rotated position. The rotational control of the rotation corrector can be designed to exert both rotational torque and compressive forces on a tibia to regulate both postero-lateral rotatory instability (PLRI) and antero-medial rotatory instability (AMRI).

In one embodiment, the invention can rotate a user's lower leg, including the user's tibia, during leg training exercise. The invention can include a cuff that can surround and tighten around a tibial portion of a user's leg to secure the tibia. The cuff can be connected to two rotational drums positioned parallel and on either side (e.g. to the left and to the right) of the user's tibial leg portion by a sheet attached to both the cuff and the drums. The drums can be rotated by means known in the art, and can include by the user utilizing appropriate leg muscles or by the user rotating a vertical bar rotatably connected to a rotational drum. The flexion and extension range can be limited by any means known in the art, including by user-controlled isokinetic testing machines or by range limiter devices. For example, the vertical bar can traverse a passage formed within a range limiter so that rotation of the vertical bar does not rotate the range limiter. Instead, the range limiter can rotate with leg extension and flexion. A load transmission bar for transmitting exercise resistance for the user's leg to overcome during leg flexion/extension can connect to a range limiter. The range limiter assembly can have any shape. In one embodiment, the range limiter can be disk-shaped.

In a further embodiment, one or both range limiters can include a range limiter track through which an immovable range limiter bar can extend, thereby constraining the range of leg flexion and/or extension during exercise. The flexion/extension range can be further controlled by range limiter pins positioned at variable locations within a range limiter track. In one embodiment, a plurality of passages can extend through the range limiter so that a range limiter pin placed within one or more of the plurality of passages can obstruct the range limiter track, thereby limiting the leg's range of motion. The range limiter track can be any portion of a notional circle centered on the center of rotation of the range limiter assembly. Preferably, the portion can be between 110° to 130° of a notional circle, more preferably approximately 120°, wherein approximately 120° can be between 115° to 125°. A series of equally spaced pin receiving passages within a 1200 range limiter track can control extension/flexion in 5° increments. Thus, by varying the number of passages to the range limiter track, the incremental control can be varied.

In another embodiment, the invention can include a load transmission disk connected to the load transmission bar. Load transmission disks are known in the art and can be circular, resulting in constant exercise resistance throughout various leg flexion/extension positions, or non-circular so that exercise resistance can be varied throughout various leg flexion/extension positions. U.S. Pat. No. 4,600,196. In a preferred embodiment, the load transmission disk can be reversible so that one piece of exercise equipment can be used for both leg extension (for exercise of quadriceps muscle) and leg curl (for exercise of hamstring muscles). A reversible load transmission disk permits the exercise resistance means to be connected either posterior or interiorly to the knee joint axis. The connection can be made by use of a load transmission belt hooked to handles on the load transmission disk located posterior or anterior to the knee joint axis. Means for providing exercise resistance are known in the art and can include weight stacks, elastic cords, springs, hydraulic valves, cylinders, pistons and the like, and brakes. In one embodiment, the exercise resistance means can be generated by a weight stack.

A further embodiment of the invention can be a cuff that can include a calf pad, a left shin pad, and a right shin pad. The right shin pad can comprise a plurality of pads wherein each of the plurality of pads can be positioned at a different vertical distance from the knee joint. In a preferred embodiment the right shin pad comprises three such pads. The left and right shin pads can be connected to opposite ends of the calf pad by an elastic material to provide support as the user inserts a lower leg into the cuff. Each pad can also be connected to the sheet so that rotation of the rotational drums can generate a rotational, as well as compressive, forces on the tibia secured by the cuff.

To assist in generating forces suitably positioned on the tibia, a series of straps, with at least one strap for each pad of the right shin pad, can be connected to the end of the calf pad to which the right shin pad is connected. Strap guides can be connected to the plurality of right shin pads to guide the straps from the calf pad, along the outward-facing side the right shin pad, and through the sheet. The straps can traverse through the sheet by a series of straps holes in a region of the sheet to which the left shin pad can be connected. These straps can be tightened to secure the cuff around the tibial portion of the lower leg to reduce or eliminate slippage between the cuff and leg. The sheet can be discontinuous between the right and left shin pad-connecting region to facilitate cuff tightening by the belts for various sized legs.

A fastener assembly can secure the tightened belts to the outward-facing surface of the sheet. (For example, the belts can traverse holes through the sheet, loop through belt rings connected to the outward-facing sheet surface, and fasten back on itself by fastening means, including velcro, buckles, snaps and the like. The sheet can also contain fastening means to connect the discontinuous portion of the sheet during leg exercise.

The invention can also include a resistance adjustment pin and resistance means connected to the resistance adjustment pin and the range limiter assembly to control the amount of force required to rotate the tibia. For example, the resistance adjustment pin can be used to lock the lower leg at a given rotational angle so that leg rotation remains constant throughout extension/flexion. The resistance adjustment pin can also be used so that the user can vary leg rotation by overcoming a user-controlled force. Alternatively, resistance adjustment pin can be set so that no force is required to rotate the leg. The leg rotation in a free-to-rotate environment can be controlled by the user rotating the vertical bar by any means known in the art, including by a handle connected to the vertical bar.

Resistance means can be generated as known in the art and can include elastics, springs, hydraulics or a brake. In one embodiment, a rubber brake attached to one end of the resistance adjustment pin can contact the vertical bar inside the range limiter disk so that tightening the pin can increase the friction between the vertical bar and the rubber brake, thereby increasing the force required to rotate the lower leg. The resistance adjustment pin can be connected to the free end of the vertical bar so that adjusting the pin increases the resistance between the resistance means and the vertical bar. Alternatively, one end of the resistance adjustment pin can be positioned on the outside of the range limiter, with a portion of the pin traversing a passage through the range limiter so that a rubber brake connected to the pin's other end can contact the vertical bar within the range limiter. Tightening the resistance adjustment pin can increase the rotational resistance; loosening the resistance adjustment pin can decrease or eliminate the rotational resistance.

The invention can also be for leg treatment exercise in a clinic or the user's home. In this embodiment, the invention can comprise a cuff, a pair of rotational drums positioned on either side of the cuff, and a sheet connecting the cuff to the rotational drums. A vertical bar can be operably connected to each of the drums so that rotation of the vertical bar rotates the rotational drum. The vertical bar can traverse a passage through a rotational assembly. In a preferred embodiment, each vertical bar traverses a passage through a rotational assembly. In a further embodiment, a treatment connection bar connects each of the rotational assemblies to provide stability to the device. The treatment connection bar can be contained within a thigh rest support. The thigh rest support can provide for comfortable positioning of the user's upper leg during exercise. The device can also include a base board attached to the thigh rest support and a belt attached to the base board, thereby providing a means to secure the device to a treatment table, or other piece of furniture such as a user's bed. Exercise resistance can be generated by an elastic belt connected at one end to hooks attached to each of the rotational drums, and at the other end to the legs of the furniture to which the device has been attached (e.g. a treatment table). Alternatively, exercise resistance can be generated by connecting one of the rotational assemblies to an isokinetic-testing machine.

The invention also provides methods for exercising a user's leg using any of the claimed devices. The method includes inserting a lower tibial portion of the user's leg into a lower leg tibial holder, rotating the lower leg into a fixed rotational angle, and flexing and extending the rotated leg. The method of exercise can be particularly useful in treating patients that have malaligned knee joints, including those with an osteoarthritic knee.

The invention used in concert with weight training exercise equipment can: (1) allow the knee to regain normal rotational range of motion and correct the rotational malalignment of the knee, (2) allow the knee to regain the muscle function of rotational control, (3) restore the range of motion and muscle strength of the knee, and (4) correct three-dimensional knee malalignment. The invention can be utilized by the younger population, including athletes as well as others, to correct rotational malalignment and can be used in physical therapy clinics, athletic training facilities as well as fitness centers.

The invention can also be used in treatment equipment to help correct rotational malalignment. With a resistance provided by an elastic means, the treatment equipment can provide sufficient resistance for strengthening the quadriceps muscles without the need for any other load generating means, including, for example, a weight stack. This treatment equipment can be used in physical therapy clinics or in the patients' home for their personal use

### BRIEF DESCRIPTION OF THE DRAWINGS

FIG. 1 is one embodiment of a rotation corrector. FIGS. 1A-1B illustrate the rotation corrector assembly for the training equipment and for the treatment equipment, respectively. FIG. 1C shows a horizontal dissection of the rotation corrector, and FIG. 1D illustrates rotation of the rotation corrector to rotate a tibia and regulate PLRI and AMRI. FIGS. 1E-1H show a cuff that surrounds the tibia that can be secured and attached to a sheet, and fastened around the lower leg, thereby imparting rotational and compressive forces on the tibia. FIG. 1E is a cross-section through the cuff. FIG. 1F is a frontal view of the cuff and sheet. FIG. 1G shows the cuff without the sheet. FIG. 1H shows how the two front portions of the cuff shown in FIG. 1F can connect to each other to secure the lower leg.

FIG. 2 shows how the rotation corrector can be incorporated into training equipment. FIG. 2A is a front perspective view and FIG. 2B is a perspective from a user sitting in the training equipment's seat.

FIG. 3 is a detailed view of a seat and thigh-rest assembly. FIG. 3A shows a thigh-rest assembly in a slide-in position. FIG. 3B shows a thigh-rest assembly in a slide-out position. FIG. 3C is a side view of a seat and thigh-rest assembly.

FIG. 4 shows one embodiment of a range limiter. FIG. 4A shows a plan view of a range limiter. FIG. 4B shows the placement of a range limiter relative to a rotation corrector. FIG. 4C shows a perspective view of a range limiter with a range limiter bar and load transmission bar.

FIG. 5 shows an embodiment of a load transmission system. In FIG. 5A the load is connected posterior to the knee for knee extension resistance. In FIG. 5B the load is connected anterior to the knee for knee flexion resistance.

FIG. 6A shows one embodiment of the treatment equipment. FIG. 6B shows the treatment equipment strapped to a treatment table.

FIG. 7A is a perspective view, from below, of the treatment equipment of FIG. 6. FIG. 7B shows the treatment equipment hooked to a treatment table by an elastic belt to generate load during treatment. FIG. 7C is a detailed view of one embodiment of an elastic belt used to generate exercise resistance.

FIG. 8A shows one embodiment where the rotation corrector can be attached to an isokinetic strength-testing machine. FIG. 8B shows a support pole. FIG. 8C shows a front view of a rotation corrector attached to an isokinetic testing machine and a support pole.

### DETAILED DESCRIPTION OF THE INVENTION

The invention may be further understood by the following non-limiting examples. Although the description herein contains many specificities, these should not be construed as limiting the scope of the invention but as merely providing illustrations of some of the presently preferred embodiments of the invention. For example, thus the scope of the invention should be determined by the appended claims and their equivalents, rather than by the examples given. All references cited herein are hereby incorporated by reference to the extent not inconsistent with the disclosure hereof.

The present invention can be used to treat a variety of knee pathologies involving rotational malalignment of the knee joint, including osteoarthritis, traumatic injury, meniscal injury, articular cartilage and ligament injury as well as abnormalities arising from rheumatoid arthritis. “Rotational alignment” is used to describe the amount the tibia is circumferentially rotated compared to the femur. The rotational alignment of the tibia varies with flexion and extension. See PCT/US20041041304, “Non-Surgically Correcting Abnormal Knee Loading” filed Dec. 10, 2004. Rotational alignment can be described in terms of the direction of the foot, in a locked position, with respect to the knee. When the foot and knee line up, the knee is rotationally aligned. When the tibia is rotated such that the foot rotates in an outward direction from the body's center, the knee (or tibia) is “externally rotated.” If the tibia rotates in the other direction, toward the inside of the body, the knee is “internally rotated.” “Malalignment” refers to a tibia with excessive rotational angle so that normal knee function and structure may be presently impaired or at risk of future impairment. Flexion, extension or flexion/extension refers to the angle defined by the tibia and femur. A straight leg is a fully extended leg and a leg that is going from straight to flexed is undergoing flexion. A leg that is being straightened from flexion is said to be approaching extension.

Rotational malalignment is multi-factorial. Components of rotational malalignment are a combination of translation and rotation of the tibia, relative to the femur. The ligaments normally restrict tibial translation, but if any are damaged the tibia may move (i.e. translate), relative to the femur, forward (anterior), backward (posterior), inside (medial) and outside (lateral). The rotational malalignment can cause an abnormal loading pattern, which can accelerate degeneration of the articular cartilage in the knee joint. Therefore, the present invention can simultaneously assist in correcting the tibia's rotational malalignment and translation to reduce and correct abnormal loading across the knee joint.

A precise observation reveals that the tibia is both externally rotated and laterally translated in the varus (bowlegged) knee. This positioning of the tibia causes an abnormal pressure distribution over the tibia. In particular, the unbalanced pressure in the varus knee causes excessive compression of the medial compartment of the knee even in the non-weightbearing position. The maximum pressure on the medial compartment occurs with standing and the condition progressively worsens under continued rotational malalignment during gait. The present invention can prevent and/or alleviate this worsening by simultaneously correcting tibial translation and abnormal rotational alignment. This correction occurs by restoring the external rotation contracture (meaning loss of normal internal rotation), followed by restoring the knee range of motion and by strengthening the muscles under the corrected rotational alignment. Repeated use of this invention can help patients restore normal knee function, and the worsening process of knee OA can be delayed more effectively with an application of the knee brace disclosed in PCT/US2004/041304, “Non-Surgically Correcting Abnormal Knee Loading” filed Dec. 10, 2004, than with the use of the leg exercise equipment alone.

As used herein, “operably connected” means two or more parts are connected such that the movement of one part can affect the operation of the other part.

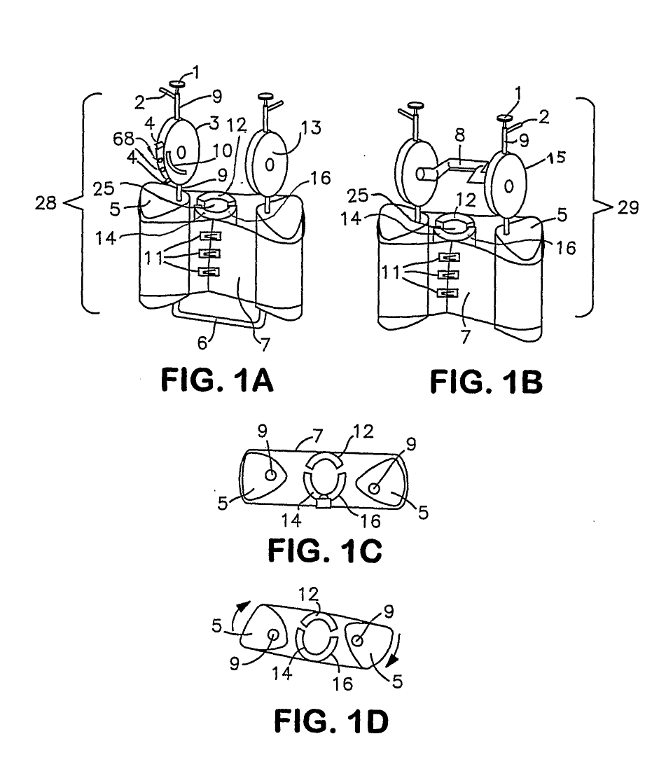

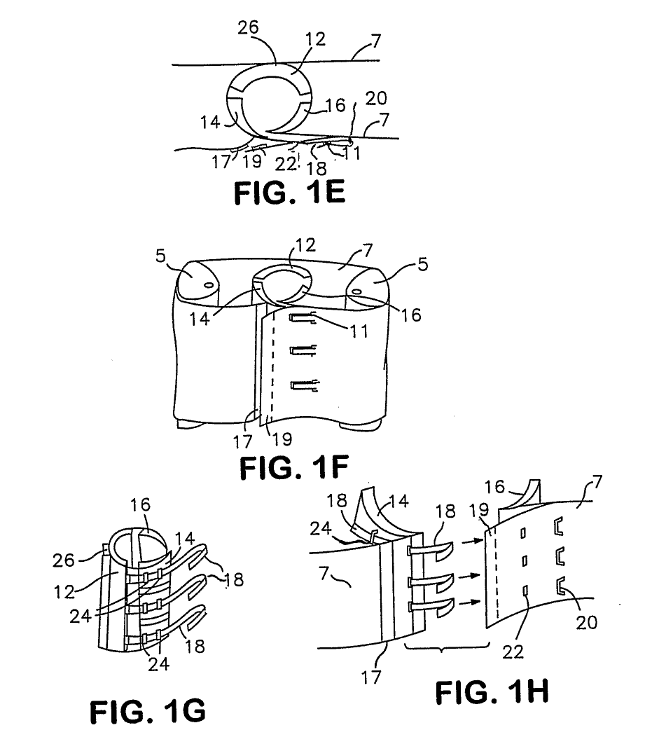

FIG. 1A shows one embodiment of a training rotation corrector assembly 28 that can be used to correct knee malalignment. A “rotation corrector” refers to a piece of exercise equipment to generate and impart a rotational force to the tibial portion of a leg to help correct the tibia's rotational malalignment. The term includes, as shown in the examples, any means to surround and secure the tibia and means to impart a rotational force to the tibia. The force can be generated by any means known in the art, including by the user rotating the device. A user's lower leg (e.g. the tibial portion of the right leg) can be placed within a cuff 25 formed by the right shin pad 14, left shin pad 16 and calf pad 12. The pads can tightly hold the lower leg in place by tightening the fastener assembly 11 over the sheet 7. Sheet 7 can be connected to a pair of rotational drums 5 and to the calf pad 12 and shin pads 14 and 16. Each rotational drum can be connected to a vertical bar 9. Thus, rotation of a vertical bar 9 can cause a corresponding rotation in each of the rotational drums 5, thereby rotating sheet 7 and a user's tibial portion of the leg. Alternatively, the user can rotate his or her lower leg by generating appropriate leg musculature forces. A rotational drum is one means whereby a rotational force can be imparted onto the tibia. The particular circumferential shape of the drum is not important so long as rotation of the drum results in movement of a sheet attached to the drum to which the cuff 25 is also attached. A connection bar 6 can connect the rotational drum pair 5 to each other to provide additional stability and ensure that drums 5 remain parallel to each other during exercise, while allowing rotation of drum 5 with rotation of vertical bar 9. The rotation corrector 28 can include means to rotate the vertical bar 9. The means to rotate the vertical bar can include a handle attached to the vertical bar or a grip that surrounds the vertical bar. For example, a handle 2 can be attached to a vertical bar 9 so that a user can rotate the handle 2 to rotate the vertical bar 9, and thereby rotate the rotational drum 5, sheet 7 and the user's lower leg. An optional resistance adjustment pin 1 can vary the force required to rotate the rotational drum 5.

The rotational sheet can be made from any material that is non-elastic and can withstand multiple exercise repetitions without failure. To prevent slippage, the sheet 7 can be attached to the rotational drum 5 by any means known in the art, including by permanent means such as screws. The sheet 7 can also be attached to the cuff 25 by any means known in the art including permanent means such as stitches or staples and temporary means, including fasteners, to permit the cuff or sheet to be removed from sheet 7 for cleaning or replacement.

In a further embodiment, the rotation corrector can also include a range limiter 3 to restrict the range of motion of the leg as the user flexes and extends the leg. The range limiter 3 can contain a range limiter track 10 and a pair of range limiter pins 4 that can be disposed within a series of range limiter pinholes 68 to control the leg's range of motion. The range limiter can contain a passage so that vertical bar 9 can traverse the range limiter assembly 3 so that the vertical bar 9 can rotate without rotating the range limiter 3. In a preferred embodiment there can be a vertical bar 9 and means for rotating the vertical bar 9 for each of the two rotational drums 5. There can be an optional second assembly 13 positioned on the opposing side of the rotation corrector. The second assembly 13 can optionally be a second range limiter 3. The range limiter can have any shape. In a preferred embodiment, the rotation corrector 28 contains two disk-shaped range limiters, wherein each range limiter disk can be positioned above each of the two drums 5, thereby improving overall stability of the rotation corrector 28 during repeated cycles of leg flexion.

FIG. 1B illustrates one embodiment of a treatment rotation corrector 29 that can be used to help correct abnormal knee rotation. The treatment rotation corrector 29 can be similar to the training rotation corrector 28. The treatment rotation corrector 29 can also include a pair of rotational drums 5 positioned on either side of a patient's lower leg, connected to sheet 7. The patient's lower leg can be surrounded and secured by a calf pad 12 and right 14 and left 16 shin pads. The rotational drums 5 can be rotated by rotation of one or more vertical bars 9, connected to rotational drums 5. Alternatively, the patient can rotate the rotational drums by actively rotating his or her tibia contained within the calf pad 12 and shin pads 14 and 16. Vertical bar 9 can traverse a passage contained within a rotational assembly 15. The rotational assembly 15 can have any shape, including disk-shaped, so long as the rotational assembly 15 can contain a passage for vertical bar 9 to traverse. A user can rotate the vertical bar 9 by rotating a handle 2 connected to the vertical bar 9. Rotating the handle 2 does not cause the rotational assembly 15 to rotate. A treatment connection bar 8 can connect a pair of rotational assemblies 15 to provide additional stability to the treatment rotational corrector. The treatment connection bar 8 can be shaped so that the connection bar 8 does not impinge on a user's upper leg during exercise. A resistance adjustment pin 1 can regulate the amount of force required to rotate the drum, and therefore, the user's tibia.

For example, resistance adjustment pin 1 in either the training 28 or the treatment 29 rotation corrector, can be connected to an associated resistance means to control the amount of force the leg must overcome to rotate the lower leg. The user can lock the lower leg at a fixed knee rotation, or the user can have the lower leg free to rotate without any resistance to rotation. Alternatively, the user can adjust the resistance so that some user effort is required to rotate the lower leg. Any means known in the art can be used to regulate resistance adjustment. The resistance means can include an elastic band. It can also include a brake such as a rubber pad in contact with an element that moves against the rubber pad when the rotation corrector is operated whereby increasing brake friction increases the force the knee rotation must overcome. “Braking connection” is used herein to refer to one element's movement effecting a change in the force required to move/rotate a second element (ranging from no force required to a force that is so large that the second element is effectively locked into position). One embodiment includes a pin with a rubber brake on one end inserted through any of the disks 3, 13, and 15 so that the rubber brake can contact the vertical bar 9. Tightening the pin can increase the friction between the rubber brake and vertical bar 9. The rotation correctors 28 and 29 can be used for passive (user rotates the vertical bar to rotate the leg), active (user rotates the leg by utilizing appropriate leg muscles against no resistance) and resistive knee therapy (user rotates leg against resistance).

FIGS. 1C-1H are more detailed drawings of a cuff used to secure a lower leg tibial portion and rotation of the lower leg by rotation of drums 5. The leg can be secured by any means known in the art including a cuff to surround the leg. The cuff can be flexible and cushioned to provide comfort for the user's leg. Alternatively, the cuff can be more rigid, made of plastic or metal to more securely hold the user's leg in place. The cuff can also comprise a combination of rigidity and cushioning to maximize the ability to hold the leg in place while also increasing the surface area in contact with the leg to avoid pain by leg compression. For instance, the cuff can contain a metal plate surrounded by soft material such as polyurethane foam and covered by artificial leather for coating. Also provided are means to secure said cuff around said leg including, for instance, belts and hooks with adjustable positions to secure the cuff around various-sized legs. Velcro may also be used as means to secure the cuff around the leg. The cuff need not be in one piece. A cuff comprised of multiple pieces to surround the leg provides a means to control the location of the force generated on the tibia.

The cuff can contain a calf pad 12, right shin pad 14 and left shin pad 16. FIGS. 1C-1D are cross-sectional drawings through the leg cuff. FIG. 1D shows the rotational drums 5 rotated in a clock-wise direction, as indicated by the arrows. Rotation of the rotational drums 5 causes the rotational sheet 7 to rotate the lower leg by exerting forces on the calf 12 and shin pads 14 and 16. Simultaneous rotation of the two rotational drums enables not only rotation of the tibia but also reduction of excessive tibial displacements associated with AMRI or PLRI. This system allows the patient to find an optimal rotational position of the tibia while executing strength training for the quadriceps or hamstrings. The rotational drums 5 can have any cross-sectional shape so long as rotation of the drums 5 can result in rotation of the lower leg contained within the leg cuff. In one embodiment, the cross-section of the drums is triangular with rounded corners.

FIG. 1E is a detailed view of the lower leg cuff. The calf pad 12 can be connected to the sheet 7 by connection 26. The connection can be of any means known in the art that firmly attaches the calf pad 12 to the sheet 7. This can include permanent means such as stitches, adhesives, one-piece molding, or temporary means such as fasteners, velcro, buckles, zippers and the like. The shin pads can be composed of two parts, right shin pad 14 and left shin pad 16 (for when the right leg is inserted in the lower leg cuff). The left shin pad 16 can be one large pad to give a pressure on the left front side of the shin, which can be attached to sheet 7. The right shin pad 14 can be composed of a plurality (preferably 3or 4) of small pads (FIG. 1G), which permit adjustment of tightness as well as circumferential adjustment to accommodate different lower leg sizes. Belts 18 can be attached to the front part of the calf pad 12 and travel on the outside of the right shin pad 14 (see FIGS. 1G-1H), which give a compression force toward the right front side of the shin. In the cuff front, the right pad 14 can be attached to the sheet 7 as shown in FIG. 1H, and holes 22 can be made in the elastic sheet 7 for the belts 18 to travel through. The portion of the belts 18 that have traversed holes 22 can be fastened to the part of the sheet 7 attached to the left shin pad 16 by any means known in the art. In one embodiment, the fastener assembly can comprise the belts 18 traversing the holes 22 and looping around hooks 20 wherein the free end of belts 18 are then fastened by fasteners 11. The fastener can utilize any fastening means, including buttons, buckles, velcro and the like. Pulling the belts 18 causes no motion on the right shin pad 14 because the belts 18 do not directly connect with the right shin pad 14. The belts 18 go through belt guides 24 so that tightening of the belts 18 can cause a compressive force on pad 14.

FIG. 1H shows that the sheet 7 can be discontinuous at the front of the leg, between the left 16 and right 14 shin pads. Sheet 7 can have flaps to connect the left shin pad 16. The sheet 7 can have another flap to attach the right shin pad 14. The wedge formed by the sheet 7 and right shin pad 14 can slide into a wedge receiving portion formed by sheet 7 attaching to left shin pad 16. This is indicated by the arrows in FIG. 1H. The free end of sheet 7 attached to left shin pad 16 can contain holes 22 for the belts 18 to travel through and rings 20 to tightly fasten the belts 18. Fastening surfaces 17 and 19 can fasten to each other to securely fasten sheet 7 in place after the cuff has been secured around the lower leg. The surfaces can fasten by any means known in the art that permit temporary fastening, including velcro, buttons, and buckles. Velcro surfaces to stabilize the system to provide sufficient compression on the shin can also be positioned between the sheets and belts.

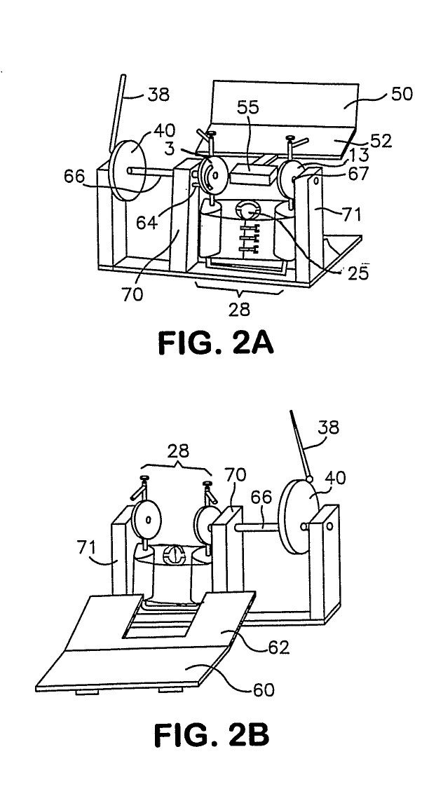

FIG. 2A illustrates an overview of training equipment with the training rotation corrector assembly 28. The rotation corrector 28 can be utilized by any leg curl and leg extension exercise equipment known in the art. The rotation corrector can be mounted between two side poles 70 and 71. During leg flexion/extension exercise, exercise resistance or load generation can be generated by any means known in the art, including those summarized in U.S. Pat. No. 4,600,196, and can be a uniform resistance weight stack, variable resistance weight stack (U.S. Pat. Nos. 3,858,873, 4,387,893, 4,456,245), elastic cords, springs (U.S. Pat. Nos. 1,750,549, 1,866,868, 3,770,267), hydraulic valves, cylinders, pistons and the like (U.S. Pat. Nos. 3,529,474, 3,784,194, 3,848,467), and brakes (U.S. Pat. No. 3,074,716). Exercise resistance or load generating means (not shown) can be connected to one end of a weight transmission belt 38. The weight transmission belt 38 can connect to a reversible load transmission disk 40. A load transmission bar 66 can impart the exercise resistance to a rotation corrector 28 by connecting to one of the range limiter 3 or optional second assembly 13. A user can sit on a seat assembly that can include a hip rest 52 and back support 50. A thigh rest 55 can adjustably extend from the hip rest 52 to facilitate placement of the user's lower leg within the cuff 25 of the rotation corrector 28. A range limiter bar 64, connected to side post 70, can limit the leg's range of motion by interacting with the range limiter 3. To improve rotation corrector 28 stability, the second assembly 13 can rotatably connect to the second side post 71 by a bar 67. The second assembly 13 can be a range limiter 3 with a corresponding range limiter 64.

FIG. 2B shows a view of exercise equipment from a user's sitting position to illustrate a platform 60 and footrest assembly 62 for the foot whose tibia is not being exercised. The training rotation corrector 28 can control tibial rotation as well as applying exercise resistance to the lower leg during flexion and/or extension. The seat, platform and footrest are designed for good accessibility by the patient and for stabilization of the patient's body during exercise. The platform 60 allows safe steps during the patient's access to an appropriate position in the seat. The footrest 62 provides secure positioning of the foot of the leg not being exercised, and the counter force from the footrest allows the patient's body to be maintained in a comfortable and stabilized position during exercise.

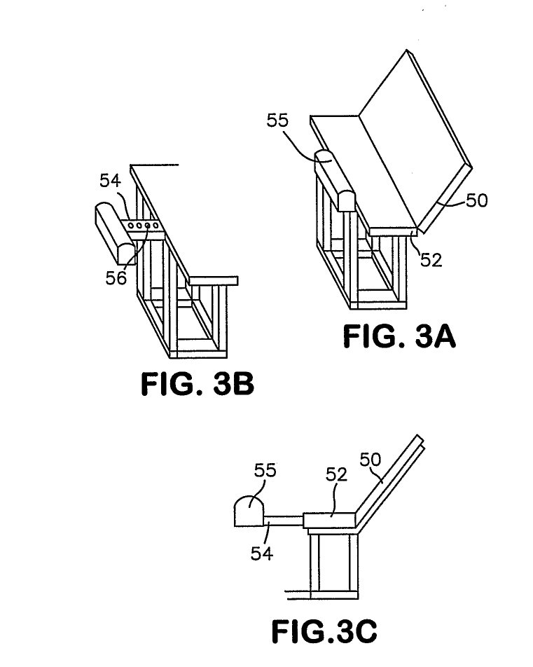

FIG. 3 illustrates the structure of the seat. The back support 50 can be tilted from the horizontal line, preferably approximately 50°, to allow a comfortable seated positioning for the patient. The hip rest 52 can be flat and horizontally aligned, and can be wide enough to allow appropriate positioning for exercising both right and left knees. The sliding thigh rest 55 can be used to stabilize the thigh of the leg being trained at its slide-out position (FIG. 3B), whereas it slides in for better patient access to the training position (FIG. 3A). A thigh rest regulator means can control the distance the thigh rest 55 can be extended from the hip rest 52. In one embodiment the regulator means can be a sliding thigh rest bar 54 with holes 56 for controlling the thigh rest 55 distance from the edge of the hip rest 52. FIG. 3C is a side view to illustrate the height of the sliding thigh rest, which can put the thigh at 20° angle, relative to horizontal, so that knee reaches 110° at its resting position (tibia being vertical) under the effect of gravity.

The seat can be designed for proper positioning of the patient, good accessibility, and ease of use in inserting the lower leg and aligning the knee onto the machine. The sliding thigh support provides better accessibility when it is in the slide-in position (FIG. 3A), and an appropriate thigh support when it is in the slide-out position (FIG. 3B). The height of the sliding thigh rest provides a tilt of the thigh, allowing increased flexion angle of the knee at its resting position under gravity.

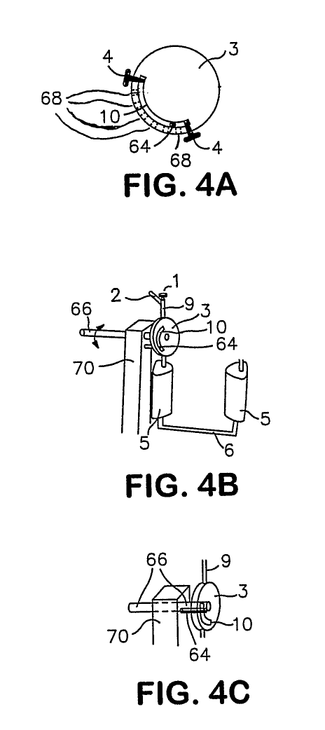

FIG. 4A is a cross-section of the range limiter 3. The range limiter 3 can contain a track 10 through which a range limiter bar 64 can be disposed. FIG. 4B shows a range limiter bar 64 rigidly connected to side post 70 for controlling the leg's range of motion. The range limiter bar 64 is stationary and does not connect to the range limiter assembly 3, but instead the range limiter assembly 3, along with track 10, rotates with leg flexion/extension. Thus, the maximum flexion or extension of the knee can be constrained by the range limiter bar 64 impinging on either end of track 10. In addition, range limiter pins 4 can provide a means of further controlling the maximum flexion/extension. FIG. 4A depicts a series of range limiter pinholes 68 (shown as dashed lines) positioned at different locations along the track 10. Placing pins 4 in different pin hole locations 68 provides a way to further constrain the rotational range of the range limiter assembly 3, thereby constraining the extension/flexion range of the user's knee joint. Pin holders, and pinholes can be located anywhere on or through the range limiter assembly 3, so long as the pins 4 can control the range of extension/flexion. FIG. 4A shows one embodiment where the range limiter pinholes 68 can be passages in a radial direction from the circumferential edge of a range limiter disk 3 through to track 10.

Track 10 can be a portion of an imaginary circle, centered on the center of rotation of the rotation limiter assembly 3, with an arc length corresponding to the typical maximum range of extension/flexion for a user's knee joint. The maximum range of motion for leg exercise is approximately 120 degrees. Track 10 can be formed completely through the range limiter 3. Alternatively, track 10 can be etched only part way through a range limiter 3 to form a channel. A vertical bar 9 can traverse a passage formed within a range limiter 3, without affecting or impinging on track 10. The range limiter 3 does not rotate when the vertical bar 9 rotates, but instead rotates with rotation of the user's knee joint.

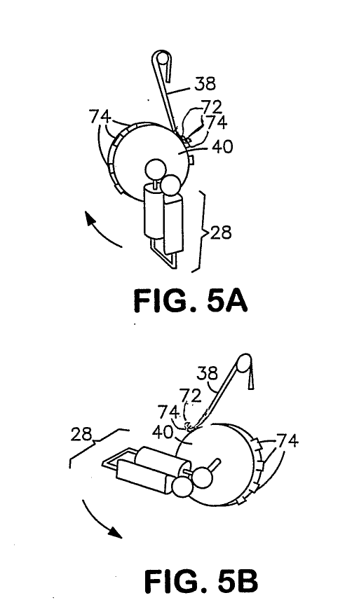

FIG. 4C is one embodiment where a load transmission bar 66 traverses a passage (shown as dashed lines) through a side post 70 to rigidly connect with a range limiter disk 3. Alternatively, the load transmission bar 66 and side post 70 can be constructed and/or shaped so that bar 66 need not traverse a passage contained within post 70. The load transmission bar 66 can rotate with range limiter 3 (as indicated by the arrow in FIG. 4B) and impart an exercise resistance by connecting to a load transmission disk 40, as shown in FIG. 5B.

FIGS. 5A-5B show one embodiment of a load transmission disk 40 used in a reversible load transmission system. A reversible load transmission system allows for changing the load direction to provide both knee extension and knee flexion exercises in one piece of equipment. The load from a weight stack system can be transmitted through a belt 38 to a load transmission disk 40. The belt 38 can have a hook 72 at one end. The hook 72 can connect to one of a plurality of handles 74 on the load transmission disk 40. The hook 72 can be connected to a handle 74 located posterior to the axis of the disk for knee extension (“leg extension”) exercise (FIG. 5A). For knee flexion (“leg curl”) exercise, the hook 72 can be connected to a handle 74 located anterior to the axis of the disk, as shown in FIG. 5B. The arrows in FIG. 5 indicate the direction of leg movement to overcome exercise resistance. To generate variable exercise resistance wherein the resistance can change with flexion/extension, the load transmission disk can have a non-uniform radius.

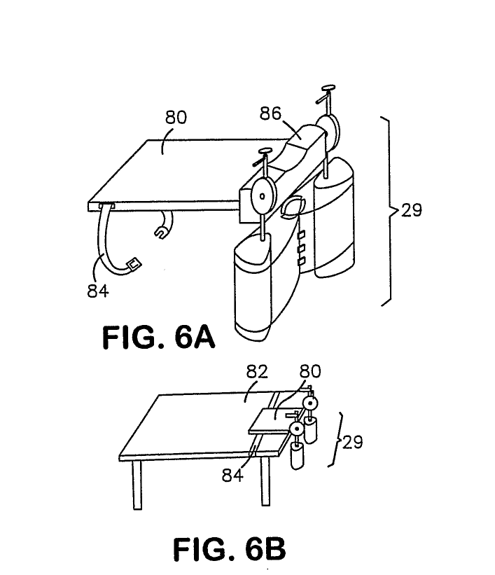

FIG. 6A is a perspective view of one embodiment of the treatment rotation corrector 29 used in treatment as well as rehabilitation equipment. The rotation corrector 29 of FIG. 1B can also include thigh support 86 to encompass the treatment connection bar 8 (shown in FIG. 1B) and provide support and comfort to the user's upper thigh leg portion. For embodiments that include a thigh support 86, the connection bar depicted in FIG. 1B can be straight. The treatment rotation corrector 29 can also include means to secure the corrector 29 to a treatment table, bed, or the like to provide stability. In one embodiment, the means can include a base board 80 and treatment board belt 84. FIG. 6B shows the base board 80 and belt 84 connected to a treatment table 82. The treatment equipment can be designed to be compact, light and portable for use on a treatment table in clinics or on furniture at clinics or patients' homes.

In terms of functionality, the treatment equipment depicted in FIG. 6 has similar functionality with the training equipment in that the tibial portion of a user's leg can flex/extend under a controlled rotation. However, the treatment equipment can be portable in that additional equipment (e.g. seat, side posts, load transmission disk and belt) associated with the training equipment depicted in FIG. 2 is unnecessary.

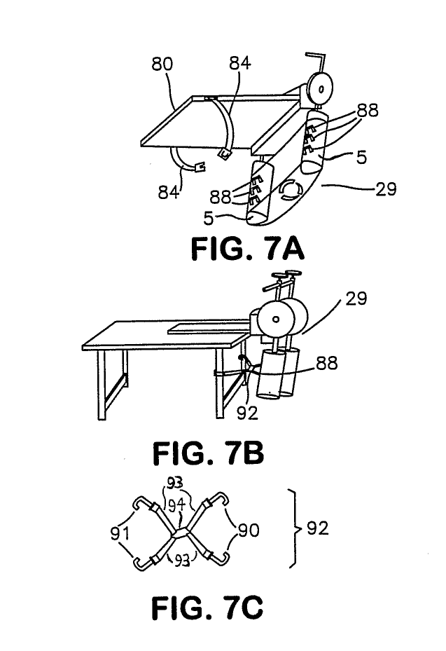

FIGS. 7A-7C illustrate one embodiment of a load application system for use with treatment equipment. FIG. 7A is a perspective view from below the base board 80 of the treatment rotation corrector shown in FIG. 6A. The rotational drums 5 have one or more handles 88 on their hind surfaces, to provide connection with hooks 90 of a specially designed elastic belt 92 (FIGS. 7B-7C). One embodiment of an elastic belt is shown in FIG. 7C, allowing gradual increase in the resistance as the elastic belt extends during knee extension exercise. The elastic belt 92 can contain four elastic bands 93 with each end of the four bands connected to a central ring 94. One hook pair 90 can connect to the drum handles 88 and the other hook pair 91 can connect to the treatment table or other furniture. The elastic properties of the rubber bands (e.g. Young's Modulus) can be manipulated by means known in the art, including by varying polymer composition and curing time.

If the hooks 90 are connected with handles 88 that are positioned higher up the drums, the force generated by the elastic band 92 can generate a proximal force on the tibia, providing posterior shear force and preventing anterior shear of the tibia. This can be useful when treating patients with anterior instability of the knee.

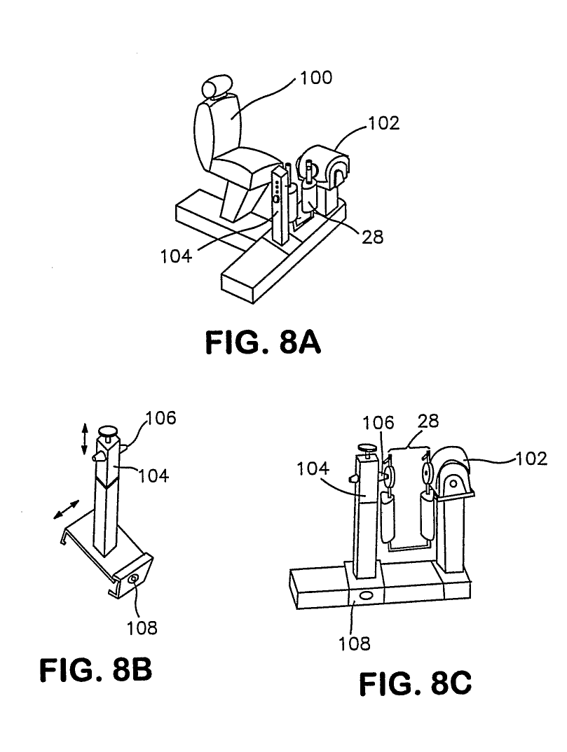

FIGS. 8A-8C illustrate a rotation corrector 29 attached to an isokinetic strength testing machine 102, such as BIODEX® System 3, which evaluates knee strength under a variety of physical conditions. For clarity, the rotation sheet and cuff are not shown in FIGS. 8A and 8C. The isokinetic strength testing machine can provide controllable range of motion and exercise resistance, so the rotation corrector 28 in FIG. 8A need not contain a range limiter and separate exercise resistance components. The rotation corrector 29 can be connected to a positionable side post 104. The positionable side post 104 can include a mounting means 106 to connect the post 104 to the rotation corrector 29. The post 104 can move in two directions, up and down to vary the rotation corrector 29 height from the ground, and horizontally by bracket 108 to connect and disconnect the rotation corrector 29 to and from the isokinetic strength testing machine 102. The devices of the present invention can be used in concert with such isokinetic strength machines to measure and evaluate rotational alignment and the effects of rotational alignment on knee strength. In a healthy knee, rotational alignment does not significantly affect the strength of the knee. In contrast, the joint pressure distribution associated with rotational malalignment can significantly affect the strength because an excessive pressure on the affected or damaged cartilage may disturb a normal kinematics of the knee joint. The result can provide the patient with information about the optimal rotational alignment as well as deteriorating positions for knee strength exercises.

For clarity, only right leg exercise was discussed. The principles disclosed in this invention are also applicable for left leg exercise.

1. A device for rotating a user's lower leg during leg flexion and extension exercise comprising:
a. a cuff to securely surround the tibial portion of a user's leg;
b. two rotational drums positioned on each side of said cuff;
c. a sheet attached to each of said rotational drums and said cuff so that rotation of said drums imparts a rotational force to the tibial portion of the leg;
d. means for rotating said drums;
e. means for limiting range of said flexion and extension;
f. means for generating an exercise resistance during leg flexion and extension.

2. The device of claim 1 further comprising a bar connecting the rotational drums so that said rotational drums remain parallel to each other during exercise.

3. The device of claim 1 wherein means for rotating said drums comprises:
a. a vertical bar rigidly connected at one end to one of said rotational drums to rotate said rotational drum.

4. The device of claim 3 wherein means for limiting range of said flexion and extension comprises:
a. a range limiter having a passage in which at least a portion of said vertical bar is disposed such that said range limiter assembly does not rotate with rotation of said vertical bar.

5. The device of claim 4 wherein the range limiter further comprises:
a. a range limiter track;
b. an immovable range limiter bar extending into said range limiter track to limit rotation of said range limiter assembly.

6. The device of claim 5 wherein the range limiter further comprises:
a. a plurality of pin-receiving passages for receiving a range limiter pin, so that the range limiter pin can constrain the rotational range of the range limiter assembly.

7. The device of claim 5 wherein the range limiter track is an arc of approximately 120° of a notional circle centered on the center of rotation of said range limiter.

8. The device of claim 5 further comprising:
a. a load transmission bar connected to said range limiter for transmitting exercise resistance.

9. The device of claim 8 further comprising:
a. a reversible load transmission disk connected to the load transmission bar to transmit exercise resistance to said range limiter.

10. The device of claim 9 wherein the exercise resistance is for leg extension exercise.

11. The device of claim 9 wherein the exercise resistance is for leg curl exercise.

12. The device of claim 9 further comprising means for generating exercise resistance.

13. The device of claim 12 wherein the means for generating exercise resistance is a weight stack.

14. The device of claim 1 wherein said cuff comprises:
a. a calf pad connected to said sheet, said calf pad having a first edge and a second edge;
b. a first shin pad connected to said sheet and connected to said first edge of said calf pad;
c. a second shin pad connected to said sheet and connected to said second edge of said calf pad, wherein said second shin pad comprises:
i. a plurality of pads;
ii. a plurality of belt guides connected to said plurality of pads;
d. straps connected to said second edge of said calf pad that traverse through said belt guides, wherein pulling the belts tightens the cuff around the tibial portion of a leg;
e. a fastener assembly for securing the straps to the sheet.

15. The device of claim 1 further comprising:
a. a resistance adjustment pin connected to said vertical bar;
b. a resistance brake connected to said resistance adjustment pin in braking connection with said range limiter such that said resistance adjustment pin controls the force required to rotate said rotational drum.

16. The device of claim 1 further comprising:
a. a handle attached to said vertical bar so that the user can rotate said rotational drum by rotating said handle.

17. A device for rotating a user's lower leg during leg extension and flexion treatment comprising:
a. a cuff to securely surround the tibial portion of a leg;
b. a first and a second rotational drum positioned on each side of said cuff;
c. a sheet attached to each of said rotational drums and said cuff so that rotation of said drums imparts a rotational force to the tibial portion of the leg;
d. means for rotating said drums.

18. The device of claim 17 wherein means for rotating said drums comprises:
a. a first vertical bar rigidly connected at one end to said first rotational drum to rotate said first rotational drum;
b. a second vertical bar rigidly connected at one end to said second rotational drum to rotate said second rotational drum;
c. a first rotational assembly with a passage for receiving a portion of said first vertical bar;
d. a second rotational assembly with a passage for receiving a portion of said second vertical bar.

19. The device of claim 18 further comprising:
a. a resistance adjustment pin connected to said vertical bar;
b. a resistance brake connected to said resistance adjustment pin in braking connection with said rotational assembly such that said resistance adjustment pin controls the force required to rotate said rotational drum.

20. The device of claim 18 further comprising:
a. a treatment connection bar connecting said first rotational assembly with said second rotational assembly to provide stability to the device;
b. a thigh rest support that envelopes the treatment connection bar.

21. The device of claim 20 further comprising:
a. a base board attached to the thigh rest support;
b. a belt attached to the base board for securing the base board to a treatment table;
c. one or more hooks attached to each of said rotational drums for receiving an elastic belt to generate exercise resistance.

22. The device of claim 17 wherein the first rotational assembly is connected to an isokinetic testing machine.

23. A method for exercising a user's leg comprising:
a. providing a device of claim 1;
b. inserting a user's lower leg into the device of step (a);
c. rotating the lower leg by a rotational angle; and
d. flexing/extending the rotated leg under an exercise resistance to exercise the user's leg.

24. A method for exercising a user's leg comprising:
a. providing a lower leg tibial holder;
b. inserting a lower leg into the lower leg tibial holder;
c. rotating the lower leg by a rotational angle by rotating the lower leg tibial holder;
d. flexing/extending the rotated leg under an exercise resistance to exercise the user's leg.

25. The method of claim 24 wherein the user's leg comprises an osteoarthritic knee.
